(19) United States
(12) Patent Application Publication
(10) Pub. No.: US 2020/0043578 A1
(43) Pub. Date: Feb. 6, 2020

(54) Performing Predictive Patient Care Options that Improve Value Based on Historical Data

(71) Applicant: CQINSIGHTS, INC., Knoxville, TN (US)

(72) Inventor: Bruce Ramshaw, Knoxville, TN (US)

(21) Appl. No.: 16/499,499

(22) PCT Filed: Mar. 28, 2018
(86) PCT No.: PCT/US18/24803

§ 371 Date: Sep. 30, 2019

Related U.S. Application Data
(60) Provisional application No. 62/477,995, filed on Mar. 28, 2017.

(51) Int. Cl. G16H 10/60; G16H 20/00

(52) U.S. Cl. CPC G16H 10/60; G16H 20/00

(57) ABSTRACT

A method of analyzing patient health data is provided to guide treatment of a patient. The method includes: receiving patient health data from the at least one health record server on an outcome analysis server corresponding to data of an accumulated treatment process of a plurality of patients; receiving proposed treatment factors from a healthcare provider corresponding to factors identified by the healthcare provider as those corresponding to an outcome of a patient treatment process; analyzing the patient health data to identify patient health data correlating to a patient treatment process identified by the healthcare provider; correlating factors of the patient treatment process with an outcome of the patient treatment process, wherein the factors indicate one of a correlation to a positive outcome and a correlation to a negative outcome; and displaying the determined one or more patient outcome factors on a user interface.

### CROSS-REFERENCE TO RELATED APPLICATION

This application claims priority to U.S. Provisional Patent Application Ser. No. 62/477,995 for “Performing predictive patient care options that improve value based on historical data” and filed on Mar. 28, 2017, the contents of which are incorporated herein by reference in its entirety.

### FIELD

This disclosure relates to the field of healthcare. More particularly, this disclosure relates to systems and methods of analyzing electronic healthcare data to improved treatment to patients.

### BACKGROUND

Currently, patients are seen within a fragmented healthcare system. Each fragment (physician office, hospital, nurse, anesthesia group, etc.) often requires a patient to provide similar information and often these various components of a patient's cycle of care do not share information and may have missing or inaccurate information. Further, it is often difficult for a patient to access the information from these various components of care.

Recent efforts have been made to transition medical records from paper records to electronic records. In part due to financial incentives from government entities, this process has been chaotic and significant unintended harm and waste has resulted. Electronic medical record (EMR) vendors have designed software adapted to current fragments of healthcare systems and are not designed around definable patient processes (such as pathways). These products are often designed to produce documentation for coding and billing, rather than for defining and measuring the value of patient care.

Because of the fragmentation, missing and erroneous information, the focus on coding and billing and the fact that these existing EMR systems are designed as proprietary such that they do not communicate with one another, and attempts to be a one-size-fits-all solution for all varieties of diseases, patients, tests and treatments, it is difficult to understand the context of care that produces the information. Further, because no costs are associated with the process of patient care, it is impossible to define, measure or improve the value of care using current EMR information alone.

Physicians and nurses are expected to use their individual knowledge through training and experience to make testing and treatment decisions based on a patient's presentation. In addition to various individual professional decisions, testing and treatment guidelines have been implemented in various ways. These guidelines have been developed through clinical trials, consensus recommendations and through quality measure mandates, among other methods.

When a patient presents with a symptom, problem or disease, current art requires a physician to rely on information generated through reductionist clinical data (clinic trials, systematic reviews, etc.) and linear statistical analyses (linear regression, chi squared, p-values, confidence intervals, etc.). Even more recent attempts at predictive analytics use reductionist generated clinical data and expert opinion (i.e. guidelines). Recent examples illustrating the lack of value attempting to use predictive analytics using reductionist science generated data and expert opinions have been found to be extremely expensive without realizing a suitable return on the investment.

What is needed, therefore, are systems and methods of analyzing electronic healthcare data to provide effective and improved treatment to patients.

### SUMMARY

The above and other needs are met by a system and method of predictive analysis of health record data to determine factors related to an outcome of a treatment process for a patient. In a first aspect, a method of analyzing patient health data to guide treatment of a patient includes: providing an outcome analysis server, at least one user terminal in communication with the data analytics server, and at least one health record server in communication with the outcome analysis server; receiving patient health data from the at least one health record server on the outcome analysis server, the patient health data comprising data corresponding to data of an accumulated treatment process of a plurality of patients; receiving proposed treatment factors from a healthcare provider, the proposed treatment factors corresponding to factors identified by the healthcare provider as those corresponding to an outcome of a patient treatment process; analyzing the patient health data to identify patient health data correlating to a patient treatment process identified by the healthcare provider; correlating factors of the patient treatment process with an outcome of the patient treatment process, wherein the factors indicate one of a correlation to a positive outcome and a correlation to a negative outcome; and displaying the determined one or more patient outcome factors on a user interface.

In one embodiment, patient health data includes cost of treatment of a patient in the context of the accumulated treatment process of the plurality of patients, and wherein one or more patient outcome factors is based on the cost of treatment of the patient.

In another embodiment, the method further includes: receiving data from a healthcare provided corresponding to a patient undergoing treatment by the healthcare provider; iteratively updating health record data on the outcome analysis server to include data received from the healthcare provider; and predicting an outcome of treatment of the patient based on the data received from the healthcare provider and the one or more patient outcome factors determined on the outcome analysis server.

In yet another embodiment, further comprising predicting factors for improving an outcome of treatment of the patient based on the one or more patient outcome factors identified on the outcome analysis server.

In one embodiment, the method further includes: removing patient identifying information from the patient health data and the received patient outcome data; aggregating the patient health data and received patient outcome data into a common format readable on the outcome analysis server.

In another embodiment, the method further including receiving input through the user interface from a healthcare provider including outcome data of a patient treated by the healthcare provider.

In yet another embodiment, the method further includes determining one or more patient outcome factors based on outcome data received from the healthcare provider through the user interface.

In one embodiment, the method further includes analyzing patient health data corresponding to data of the accumulated treatment process of the plurality of patients to identify additional factors correlating to one of a positive treatment outcome and a negative treatment outcome.

In another embodiment, the method further includes the step of stripping the patient health data of personally identifiable information.

In a second aspect, a system for predicting a patient treatment outcome includes: a plurality of health record data sources containing health record data, the health record data sources selected from the group consisting of electronic medical records, post-operative data received from one or more healthcare providers, and post-operative data received from one or more patients; a predictive analysis server in electronic communication with the health record data sources for receiving the health record data from the plurality of health record data sources, aggregating the received health record data into an accumulated treatment process data of a plurality of patients corresponding to the received health record data, receiving data from a healthcare provider corresponding to a proposed treatment process of the healthcare provider, analyzing the accumulated treatment process data based on data from the healthcare provider to correlate a plurality of factors with a treatment outcome of the proposed treatment process from the healthcare provider, and transmitting data related to the analyzed accumulated treatment process data to a user.

In one embodiment, the system further includes analyzing the accumulated to determine additional factors correlating to an outcome of a patient treatment process. In another embodiment, the system further includes a healthcare provider terminal in electronic communication with the predictive analysis server, wherein data is received from the healthcare provider terminal and accumulated with the received health record data.

In yet another embodiment, the system further includes a patient terminal in electronic communication with the predictive analysis server, wherein data is received from the patient corresponding to treatment of the patient.

### BRIEF DESCRIPTION OF THE DRAWINGS

Further features, aspects, and advantages of the present disclosure will become better understood by reference to the following detailed description, appended claims, and accompanying figures, wherein elements are not to scale so as to more clearly show the details, wherein like reference numbers indicate like elements throughout the several views, and wherein:

FIG. 1 shows a diagram of a predictive analysis system according to one embodiment of the present disclosure;

FIG. 2 shows a flowchart of a system and methods of predictive patient analysis according to one embodiment of the present disclosure;

FIG. 3 shows a flowchart of a system and methods of predictive patient analysis according to one embodiment of the present disclosure;

FIG. 4 illustrates a diagram of systems and methods of predictive patient analysis according to one embodiment of the present disclosure;

FIG. 5 illustrates a computing device according to one embodiment of the present disclosure; and

FIG. 6 illustrates patient outcome factors according to one embodiment of the present disclosure.

### DETAILED DESCRIPTION

Various terms used herein are intended to have particular meanings. Some of these terms are defined below for the purpose of clarity. The definitions given below are meant to cover all forms of the words being defined (e.g., singular, plural, present tense, past tense). If the definition of any term below diverges from the commonly understood and/or dictionary definition of such term, the definitions below control.

The present disclosure relates to an electronic system and related methods of collecting and analyzing predictive patient care information to give patients and healthcare entities (i.e. physicians, hospitals, payers, medical device and pharmaceutical companies) real-time value-based data of patient care options and products used in patient care. Embodiments of the present disclosure include systems and methods of electronic healthcare clinical information management, analysis, and visualization that allow for local predictive analytics and global pooling of data for additional guidance and analysis. Definable patient care processes (also referred to as “pathways”) are identified by data corresponding to a patient problem, symptom, or disease, and data corresponding to patient outcomes is analyzed to determine value in the context of patient care processes. As data accumulates, the analysis of the data may be produced at local and global levels. The analysis of data corresponding to patients and their outcomes may be used to determine appropriate testing and treatment options and to improve treatment of a patient.

Embodiments of the present disclosure further include methods of predictive analysis and continuous value-based improvement of patient care processes, including steps of collecting and analyzing electronic record data documenting process steps and value-based outcomes, including costs of care. Data may be analyzed using non-linear data analytics and local interpretation of the data to allow for identification and implementation of the data to facilitated value-based patient care process improvement.

Further aspects of the present embodiment provide for adaptive improvement in an ability to measure factors within the patient care process and to measure outcomes that determine value for each patient care process. A feedback loop improves measurement of patient factors, patient process factors, and iterative analysis and visualization provides insight to clinical users that may generate improvements on measured definitions utilized by clinical users.

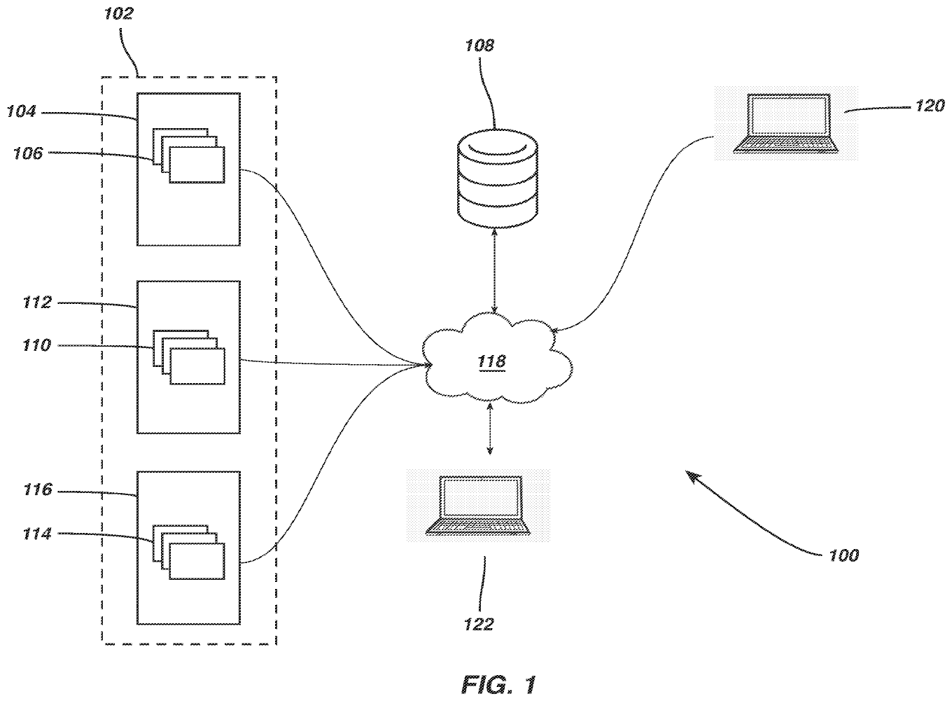

Referring now to FIG. 1, a high-level architecture of predictive analysis system 100 and external interfaces for a system for analyzing and improving patient care are shown. Embodiments of the present disclosure include sources of patient health data 102. For example, one or more health record servers 104 may servers containing patient health data corresponding to an individual, group of individuals, or entity that owns, controls, or manages electronic personal health information (“PHI”) relating to one or a plurality of patients, e.g., physicians, groups of physicians, hospitals, hospital systems, research institutions, etc. By way of example only, a healthcare practice or groups of healthcare practices employing a plurality of physicians, physicians' assistants, nurse practitioners, and office administrators may be may be associated with data on the one or more health record servers 104 for purposes of the present disclosure.

Embodiments of the present disclosure further include the transmission of electronic medical record data 106 of the one or more healthcare providers, which may include any individual employee, contractor, or agent of the one or more healthcare providers associated with the one or more health record servers 104 that uses the predictive analysis system 100 to either transmit, create, manipulate, or receive data, either on behalf of one or more healthcare providers associated with the one or more health record servers 104.

A predictive analysis server 108 stores data received from the one or more health record servers 104. Data received from the one or more health record servers 104 includes, but is not limited to, health information of a personal, and protected nature (“PHI Data”), as well as data that either contains no personally identifiable health information, or has been stripped of any PHI Data by the predictive analysis server 108 (“Non-PHI Data”).

In various embodiments, health record data may be imported to or exported from the System 116 by Users. This Data 112 may include, but is not limited to, health information of a personal, and protected nature (“PHI Data”), as well as data that either contains no personally identifiable health information or has been stripped of any PHI Data by the System 116 (“Non-PHI Data”).

With continued reference to FIG. 1, sources of health record data include electronic medical record data 106, clinical team input data 110 from a clinical team data source 112, and patient input data 114 from a patient data source 116. Clinical team input data 110 and patient input data 114 may be collected from one or more patients and healthcare providers, and preferably includes data collected from the one or more patients and healthcare providers during and after treatment of a patient. Additional data received on the predictive analysis server 108 includes healthcare literature and other publication data. Health record data also preferably includes financial data related to an overall cost of a patient treatment process, including inpatient and outpatient costs associated with patient treatment processes.

The electronic medical record data 106, clinical team input data 110, and patient input data 114 are transmitted to the predictive analysis server 108 and stored in a format such that the data is aggregated for analysis by the predictive analysis server 108. The electronic medical record data 106, clinical team input data 110, and patient input data 114 are preferably transmitted to the predictive analysis server 108 over a network 118, such as over a wired or wireless connection, the Internet, satellite, or other suitable wireline or wireless networks or combinations thereof. The received electronic medical record data 106, clinical team input data 110, and patient input data 114 are analyzed to provide history of a whole and definable patient treatment process and data related thereto.

A healthcare provider terminal 120 is in electronic communication with the predictive analysis server 108. A healthcare provider user interface is displayed on the provider terminal 120 for displaying visualized data related to processes described herein. Further, input is received from healthcare providers through the terminal 120, such as by providing input fields in the user interface for receiving data from the healthcare provider.

A patient terminal 122 may also be in electronic communication with the predictive analysis server 122 for receiving input data from a patient, the received data related to the patient's health and treatment of the patient. For example, a user interface may be displayed on the patient terminal 122

As referred to above, patient input data may be received from either the patient terminal 122 or healthcare provider terminal 120. In one example, an electronic form is displayed to the patient on the patient terminal 122 including form fields related to information corresponding to a treatment process undergone by the patient. Similarly, healthcare input data may be collected through the healthcare provider terminal 120 by presenting an electronic form on the healthcare provider terminal to collect data related to treatment of a patient.

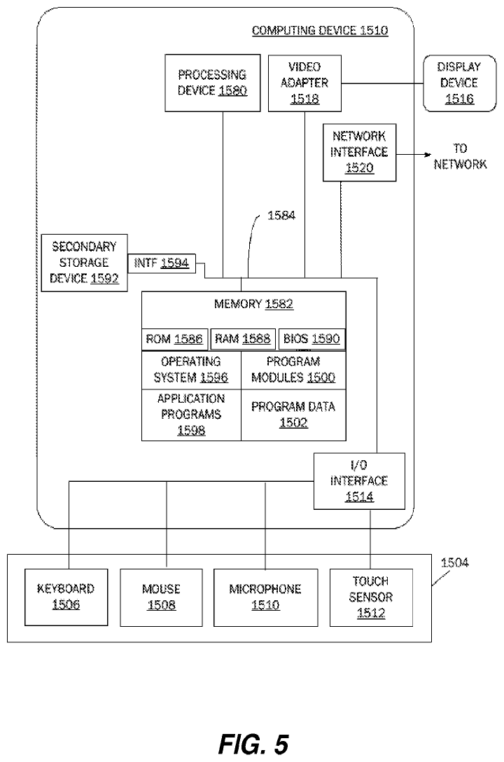

Referring now to FIG. 5, an exemplary architecture of a computing device that can be used to implement aspects of the present disclosure is illustrated, such as the predictive analysis server 108, healthcare provider terminal 120, and patient terminal 122. The computing device illustrated in FIG. 5 can be used to execute the operating system, application programs, and software modules (including the software engines) described herein.

The computing device 1510 includes, in some embodiments, at least one processing device 1580, such as a central processing unit (CPU). A variety of processing devices are available from a variety of manufacturers, for example, Intel or Advanced Micro Devices. In this example, the computing device 1510 also includes a system memory 1582, and a system bus 1584 that couples various system components including the system memory 1582 to the processing device 1580. The system bus 1584 is one of any number of types of bus structures including a memory bus, or memory controller; a peripheral bus; and a local bus using any of a variety of bus architectures.

Examples of computing devices suitable for the computing device 1510 include a desktop computer, a laptop computer, a tablet computer, a mobile computing device (such as a smart phone, a tablet device, or other mobile devices), or other devices configured to process digital instructions.

The system memory 1582 includes read only memory 1586 and random access memory 1588. A basic input/output system 1590 containing the basic routines that act to transfer information within computing device 1510, such as during start up, is typically stored in the read only memory 1586.

The computing device 1510 also includes a secondary storage device 1592 in some embodiments, such as a hard disk drive, for storing digital data. The secondary storage device 1592 is connected to the system bus 1584 by a secondary storage interface 1594. The secondary storage devices 1592 and their associated computer readable media provide nonvolatile storage of computer readable instructions (including application programs and program modules), data structures, and other data for the computing device 1510.

Although the exemplary environment described herein employs a hard disk drive as a secondary storage device, other types of computer readable storage media are used in other embodiments. Examples of these other types of computer readable storage media include magnetic cassettes, flash memory cards, digital video disks, Bernoulli cartridges, compact disc read only memories, digital versatile disk read only memories, random access memories, or read only memories. Some embodiments include non-transitory media. Additionally, such computer readable storage media can include local storage or cloud-based storage.

A number of program modules can be stored in secondary storage device 1592 or memory 1582, including an operating system 1596, one or more application programs 1598, other program modules 1500 (such as the software engines described herein), and program data 1502. The computing device 1510 can utilize any suitable operating system, such as Microsoft Windows™, Google Chrome™, Apple OS, and any other operating system suitable for a computing device. Other examples can include Microsoft, Google, or Apple operating systems, or any other suitable operating system used in tablet computing devices.

In some embodiments, a user provides inputs to the computing device 1510 through one or more input devices 1504. Examples of input devices 1504 include a keyboard 1506, mouse 1508, microphone 1510, and touch sensor 1512 (such as a touchpad or touch sensitive display). Other embodiments include other input devices 1504. The input devices are often connected to the processing device 1580 through an input/output interface 1514 that is coupled to the system bus 1584. These input devices 1504 can be connected by any number of input/output interfaces, such as a parallel port, serial port, game port, or a universal serial bus. Wireless communication between input devices and the interface 1514 is possible as well, and includes infrared, BLUETOOTH® wireless technology, 802.11a/b/g/n, cellular, or other radio frequency communication systems in some possible embodiments.

In this example embodiment, a display device 1516, such as a monitor, liquid crystal display device, projector, or touch sensitive display device, is also connected to the system bus 1584 via an interface, such as a video adapter 1518. In addition to the display device 1516, the computing device 1510 can include various other peripheral devices (not shown), such as speakers or a printer.

When used in a local area networking environment or a wide area networking environment (such as the Internet), the computing device 1510 is typically connected to a network through a network interface 1520, such as an Ethernet interface. Other possible embodiments use other communication devices. For example, some embodiments of the computing device 1510 include a modem for communicating across the network.

The computing device 1510 typically includes at least some form of computer readable media. Computer readable media includes any available media that can be accessed by the computing device 1510. By way of example, computer readable media include computer readable storage media and computer readable communication media.

Computer readable storage media includes volatile and nonvolatile, removable and non-removable media implemented in any device configured to store information such as computer readable instructions, data structures, program modules or other data. Computer readable storage media includes, but is not limited to, random access memory, read only memory, electrically erasable programmable read only memory, flash memory or other memory technology, compact disc read only memory, digital versatile disks or other optical storage, magnetic cassettes, magnetic tape, magnetic disk storage or other magnetic storage devices, or any other medium that can be used to store the desired information and that can be accessed by the computing device 1510.

Computer readable communication media typically embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are also included within the scope of computer readable media.

The computing device illustrated in FIG. 5 is also an example of programmable electronics, which may include one or more such computing devices, and when multiple computing devices are included, such computing devices can be coupled together with a suitable data communication network so as to collectively perform the various functions, methods, or operations disclosed herein.

The operation of the predictive analysis system 100 may be conducted using software, hardware, human resources, or any combination of the preceding. The predictive analysis system 100 provides a means to: continuously improve patient care; simultaneously analyze health record data received on the predictive analysis server 108; identify patient health variables based on the analyzed health record data; receive input data from patients and healthcare providers corresponding to ongoing treatment of the patient; iteratively update analysis of health record data based on input received from patients and healthcare providers; and identifying effective options for treatment of the patient based on the analyzed health record data. Patient data is analyzed as a whole—including both inpatient and outpatient data—to identify patterns within particular populations of patients and to predict variables and factors that impact a health outcome of a particular patient. Data on the predictive analysis server 108 is iteratively updated based on data continuously received from healthcare providers and patients to further identify and predict variables and factors that impact a health outcome of a patient.

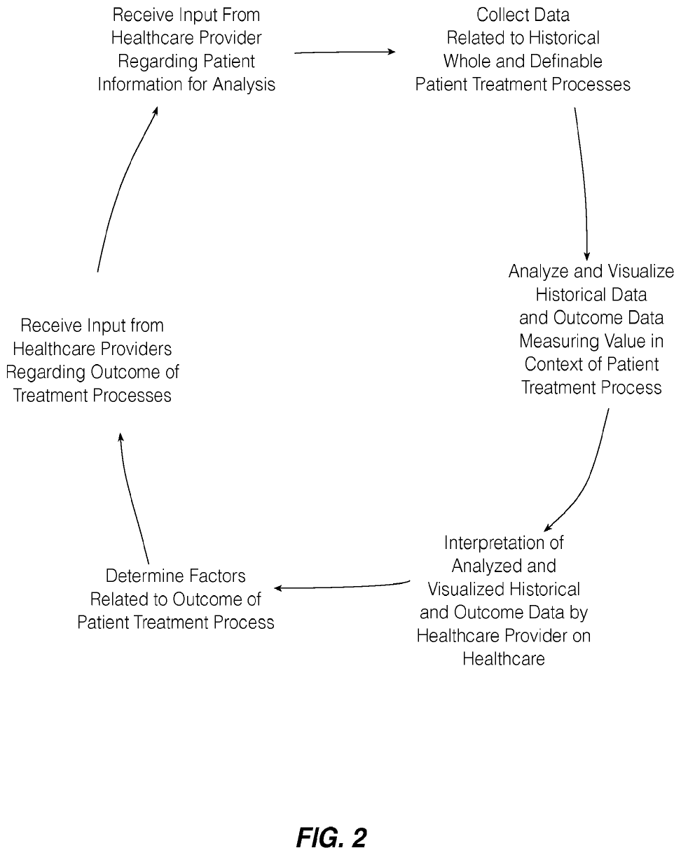

Referring to FIG. 2, processes of the predictive analysis system 100 include a feedback loop for collection and analysis of health record data that includes a whole and definable patient treatment process. Processes described herein include receiving input data from a healthcare provider regarding patient information for analysis. For example, the healthcare provider may input data related to a patient's healthcare history or other data related to the patient and a condition of the patient. Health record data is collected on the predictive analysis server 108 and analyzed in relation to input data from the healthcare provider regarding a patient. Data related to the particular patient may be visualized, such as by visualizing factors and outcome data based on data of a particular patient in relation to health record data analyzed on the predictive analysis system 100.

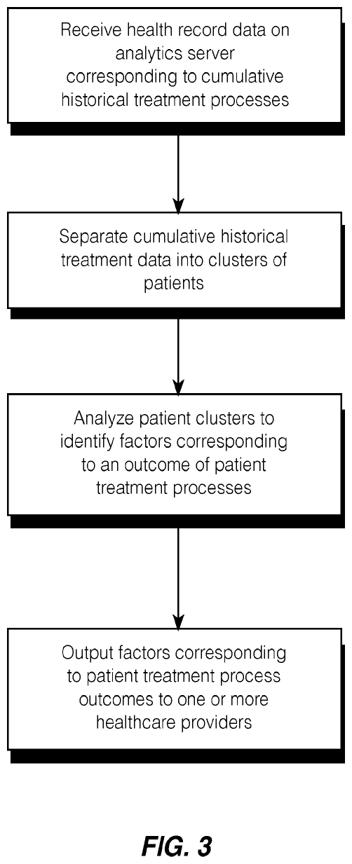

Referring now to FIG. 3, the predictive analysis server 108 analyzes health record data to determine variables or factors of groups or clusters of patients to determine outcome factors and variables related to the group or cluster of patients. In a first step, the health record data described above is simultaneously analyzed on the predictive analysis server 108 such that patients corresponding to the health record data are placed into one or more clusters or groups of patients. Those groups or clusters of patients are created based on factors identified in the health record data of the patients and their respective outcomes. For example, clusters of patients may be based on types of procedures performed on the patients. Factors are identified within the cluster of patients and correlated to the patient's outcome to identify factors and variables that provide a positive outcome of the patient based on historical health record data of an entire treatment process of patients and additional data received from healthcare providers or patients subsequent to treatment.

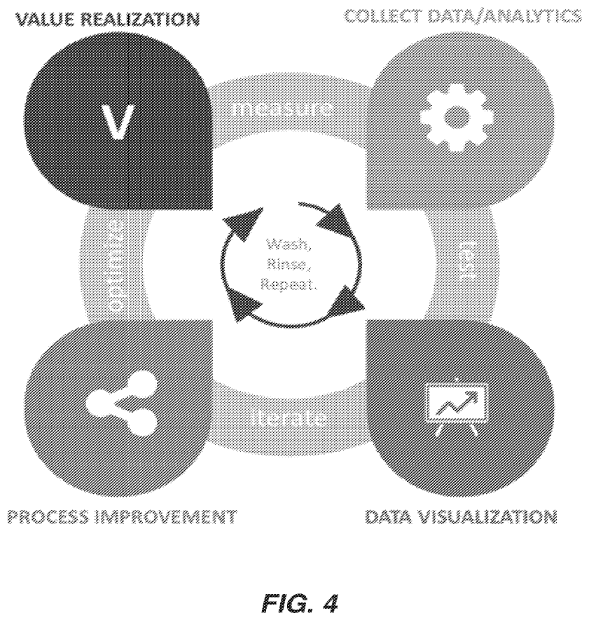

As shown in FIG. 4, health record data is collected, including data related to patient treatment and outcome as a whole. The data is analyzed and tested to identify patterns within populations of patients based on the collected health record data to predict independent variables that may impact a health outcome of a patient. The analyzed data is visualized for display to one or more healthcare providers. Input data may be received from the healthcare providers and patients to iteratively update data corresponding to an entire health of patients. Factors are identified from the data corresponding to the entire health of the patients to identify value-based factors related to treatment options for patients.

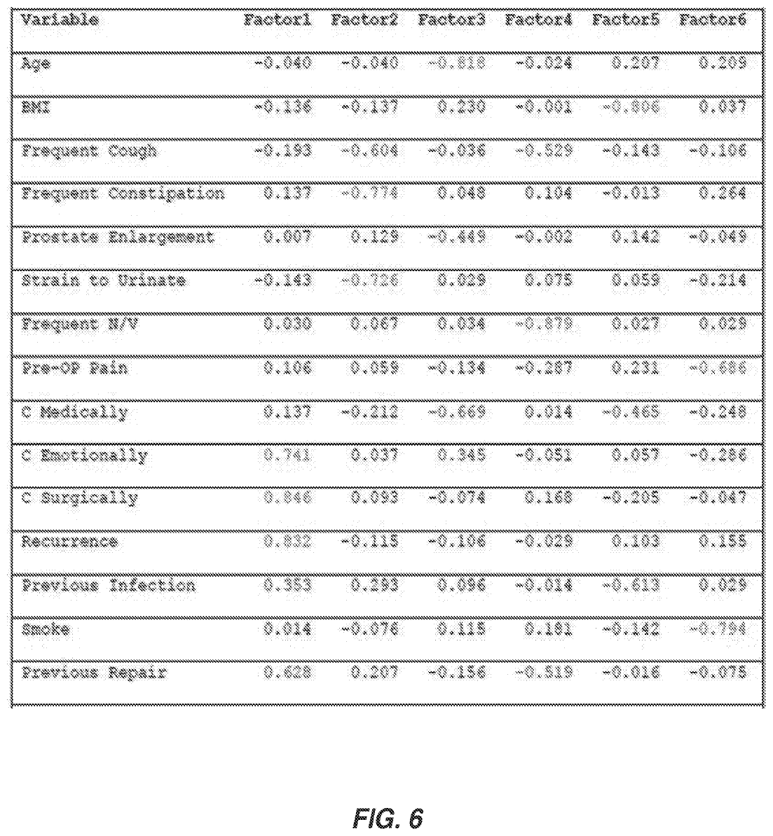

The predictive analysis system may be employed to identify factors related to patient outcome in the context of hernia patients. Specifically, factor analysis may be employed to produce a positive or negative number that is a correlation between a negative outcome and a positive outcome. The factor analysis identifies factors or variables that contribute most to an outcome of a particular process, such as the factors shown in the table of FIG. 6. For example, when the process is ventral hernia disease, factor analysis may be performed and will find that the use of drains has a highly weighted correlation to poor outcomes. Poor outcomes may be identified as increased length of stay (LOS), increased opioid use, and increased incidence of post-operative complications.

In contrast, factor analysis may reveal that for the same process, a patient's pre-operative emotional state may have an impact on the outcome of the process for the patient. Data related to the patient's emotional state may be gathered through one of the healthcare provider terminal 120 and patient terminal 122. The patient or healthcare provider may provide data to the predictive analysis server 108 related to the patient's emotional state before, during, and after a healthcare process. In the exemplary table of FIG. 6, it has been found that the emotional state of a patient preoperatively was the highest weighted modifiable factor that correlated with bad outcomes.

Data related to the analysis and identification of factors and variables may be transmitted and displayed to the healthcare provider for interpretation and action. For example, when a patient's emotional state is found to correlate to a bad outcome, the healthcare provider may implement steps to address emotional support of the patients before the healthcare process to improve the likelihood of a positive outcome of the patient.

Embodiments of the predictive analysis system 100 further may be used to define a value for an entire cycle of care of a patient. Data related to patient costs is collected on the predictive analysis server 108. In one embodiment, costs of a particular patient treatment process are estimated based on known individual procedure costs or other related data. Financial data may be transmitted from a healthcare provider, such as a hospital, to the predictive analysis server 108 for determining costs of a treatment process.

When a healthcare provider or other entity desires to determine factors related to treatment of a patient using embodiments described herein, the healthcare provider may first identify factors in a particular process that could potentially that may be believed to impact an outcome of treatment. For example, factors related to gender, BMI, number of prior procedures, and emotional state of the patient may be identified by a user as relevant factors. The predictive analysis system 100 analyzes the health record data based on these factors to identify which factors have positive and negative correlations related to patient outcome. Further, the predictive analysis system 100 may determine additional factors related to patient outcome for display to the user.

Aggregated data on the predictive analysis server 108 may be used to provide various healthcare analytics, such as financial analytics, quality reporting, and risk management. Further, patient care may be coordinated, and patients engaged to receive additional data from patients related to a treatment process. This enables a patient's outcome to be predicted based on generalized population outcomes and improves reimbursement of healthcare providers by providing treatments of value.

The predictive analysis system 100 advantageously enables the analysis of groups of patients in the context of an entire health history of the patients instead of using reductionist theories. The health record data accumulated from various data sources may be analyzed to yield actionable variables and factors for improving an outcome of a patient undergoing treatment. Further, the predictive analysis system 100 allows for the iterative improvement of the analysis based on data received from both healthcare providers and patients, thereby increasing an accuracy of the data and allowing outcome factors to be identified. Finally, the predictive analysis system 100 evaluates an outcome of patients based on cost and other factors to further improve patient treatment and the likelihood of reimbursement for the healthcare provider.

The foregoing description of preferred embodiments of the present disclosure has been presented for purposes of illustration and description. The described preferred embodiments are not intended to be exhaustive or to limit the scope of the disclosure to the precise form(s) disclosed. Obvious modifications or variations are possible in light of the above teachings. The embodiments are chosen and described in an effort to provide the best illustrations of the principles of the disclosure and its practical application, and to thereby enable one of ordinary skill in the art to utilize the concepts revealed in the disclosure in various embodiments and with various modifications as are suited to the particular use contemplated. All such modifications and variations are within the scope of the disclosure as determined by the appended claims when interpreted in accordance with the breadth to which they are fairly, legally, and equitably entitled.

What is claimed is:

1. A method of analyzing patient health data to guide treatment of a patient, the method comprising:
providing an outcome analysis server, at least one user terminal in communication with the data analytics server, and at least one health record server in communication with the outcome analysis server;
receiving patient health data from the at least one health record server on the outcome analysis server, the patient health data comprising data corresponding to data of an accumulated treatment process of a plurality of patients;
receiving proposed treatment factors from a healthcare provider, the proposed treatment factors corresponding to factors identified by the healthcare provider as those corresponding to an outcome of a patient treatment process;
analyzing the patient health data to identify patient health data correlating to a patient treatment process identified by the healthcare provider;
correlating factors of the patient treatment process with an outcome of the patient treatment process, wherein the factors indicate one of a correlation to a positive outcome and a correlation to a negative outcome; and displaying the determined one or more patient outcome factors on a user interface.

2. The method of analyzing patient health data of claim 1, wherein patient health data includes cost of treatment of a patient in the context of the accumulated treatment process of the plurality of patients, and wherein one or more patient outcome factors is based on the cost of treatment of the patient.

3. The method of analyzing health data of claim 1, further comprising:
receiving data from a healthcare provided corresponding to a patient undergoing treatment by the healthcare provider;
iteratively updating health record data on the outcome analysis server to include data received from the healthcare provider;
predicting an outcome of treatment of the patient based on the data received from the healthcare provider and the one or more patient outcome factors determined on the outcome analysis server.

4. The method of claim 3, further comprising predicting factors for improving an outcome of treatment of the patient based on the one or more patient outcome factors identified on the outcome analysis server.

5. The method of analyzing patient health data of claim 1, further comprising:
removing patient identifying information from the patient health data and the received patient outcome data;
aggregating the patient health data and received patient outcome data into a common format readable on the outcome analysis server.

6. The method of analyzing patient health data of claim 1, further comprising receiving input through the user interface from a healthcare provider including outcome data of a patient treated by the healthcare provider.

7. The method of analyzing patient health data of claim 6, further comprising determining one or more patient outcome factors based on outcome data received from the healthcare provider through the user interface.

8. The method of analyzing patient health data of claim 1, further comprising analyzing patient health data corresponding to data of the accumulated treatment process of the plurality of patients to identify additional factors correlating to one of a positive treatment outcome and a negative treatment outcome.

9. The method of analyzing patient health data of claim 1, further comprising the step of stripping the patient health data of personally identifiable information.

10. A system for predicting a patient treatment outcome comprising:
a plurality of health record data sources containing health record data, the health record data sources selected from the group consisting of electronic medical records, post-operative data received from one or more healthcare providers, and post-operative data received from one or more patients;
a predictive analysis server in electronic communication with the health record data sources for:
receiving the health record data from the plurality of health record data sources;
aggregating the received health record data into an accumulated treatment process data of a plurality of patients corresponding to the received health record data;
receiving data from a healthcare provider corresponding to a proposed treatment process of the healthcare provider;
analyzing the accumulated treatment process data based on data from the healthcare provider to correlate a plurality of factors with a treatment outcome of the proposed treatment process from the healthcare provider; and
transmitting data related to the analyzed accumulated treatment process data to a user.

11. The system of claim 10, further comprising analyzing the accumulated to determine additional factors correlating to an outcome of a patient treatment process.

12. The system of claim 10, further comprising a healthcare provider terminal in electronic communication with the predictive analysis server, wherein data is received from the healthcare provider terminal and accumulated with the received health record data.

13. The system of claim 10, further comprising a patient terminal in electronic communication with the predictive analysis server, wherein data is received from the patient corresponding to treatment of the patient.
